(19) United States
(12) Patent Application Publication
(10) Pub. No.: US 2007/0249865 A1
(43) Pub. Date: Oct. 25, 2007

(54) Process for Preparation of 4-Fluoro-Alpha-[2-Methyl-1-Oxopropyl]-Gamma-Oxo-N-Beta- Diphenylbenzene Butane Amide

(75) Inventors: Joy Mathew, Karnataka (IN); Tom Thomas Puthiaprampil, Karnataka (IN); Ravindra Chandrappa, Karnataka (IN); Sambasivam Ganesh, Karnataka (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: BIOCON LIMITED, Bangalore, Karnataka (IN)

(21) Appl. No.: 11/574,296

(22) PCT Filed: Aug. 26, 2004
(86) PCT No.: PCT/IN04/00264

§ 371 Date: Feb. 26, 2007

(51) Int. Cl. C07C 235/80

(52) U.S. Cl. 564/169

(57) ABSTRACT

A novel process for the preparation of substantially pure 4-Fluoro-α-[2-methyl-1-oxopropyl]γ-oxo-N-β-diphenyl-benzene butane amide.

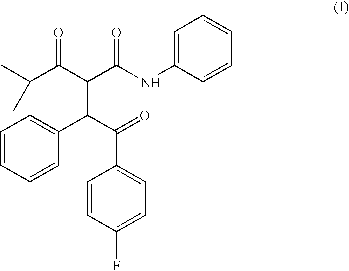

### TECHNICAL FIELD OF THE INVENTION

The present invention relates to a process for preparing 4-Fluoro-α-[2-methyl-1-oxopropyl]γ-oxo-N-β-diphenylbenzene butane amide (Formula I), a key intermediate useful for synthesis of HMG-COA enzyme inhibitor, atorvastatin.

### BACKGROUND OF THE INVENTION

U.S. Pat. No. 5,124,482 and U.S. Pat. No. 5,216,174 disclose manufacture and use of 4-Fluoro-α-[2-methyl-1-oxopropyl]γ-oxo-N-β-diphenylbenzenebutane amide for preparation of Atorvastatin was first disclosed in U.S. Pat. No. 4,681,893. Atorvastatin calcium was claimed in U.S. Pat. No. 5,273,995.

Many patent application(s)/publications disclose process for the preparation of Atorvastatin calcium viz. U.S. Pat. No. 5,003,080, U.S. Pat. No. 5,169,857, WO 01/85702, U.S. Pat. No. 5,354,772, EP 0 304 063

A key intermediate in the process for the synthesis of Atorvastatin known as “4-Fluoro-α-[2-methyl-1-oxopropyl]γ-oxo-N-β-diphenylbenzene butane amide” (Formula I) was disclosed in patents U.S. Pat. No. 5,124,482. The compound of Formula I can be further processed to get atorvastatin and the purity of the final product atorvastatin is highly dependent on the purity of the compound of Formula I.

Processes for the preparation of 4-Fluoro-α-[2-methyl-1-oxopropyl]γ-oxo-N-β-diphenylbenzene butane amide are disclosed in the patent(s)/application(s) viz. U.S. Pat. No. 5,216,174. A process for the preparation of compound of Formula I is also disclosed in a research article (J. Labelled Cpd. Radiopharm. 42, 121-127, 1999) where it is mentioned that presence of trace amounts of water during the synthesis of compound of Formula I led to the formation and impurity namely “desfluro derivative of compound of Formula I”.

The prior art processes suffer from a major disadvantage of generation of impurities like α-[2-methyl-1-oxopropyl]γ-oxo-N-β-diphenylbenzene butane amide and difluoro-α-[2-methyl-1-oxopropyl]γ-oxo-N-β-diphenylbenzene butane amide. According to the prior art literature, the reaction need to be carried out under controlled conditions (e.g.: highly anhydrous conditions) to avoid formation of the impurities. The prior art also mentions that presence water, even in trace amounts, result in the impurities. In fact desfluro atorvastatin is one of the known impurities in atorvastatin, which arises due to the presence of like α-[2-methyl-1-oxopropyl]γ-oxo-N-β-diphenylbenzene butane amide in 4-Fluoro-α-[2-methyl-1-oxopropyl]γ-oxo-N-β-diphenylbenzene butane amide, used for the manufacture of atorvastatin.

Therefore, there is a need to find alternative process, which can be used, for the preparation of 4-Fluoro-α-[2-methyl-1-oxopropyl]γ-oxo-N-β-diphenylbenzene butane amide which does not lead to the formation of these impurities and does not require the controlled conditions to be maintained during the synthesis.

The instant invention provides a solution to the above-mentioned problems and provides a more preferred alternative to the prior art processes.

The objective of the present invention is to provide an alternative, industrially scalable process for the synthesis of substantially pure compound of Formula I which can be used to get substantially pure atorvastatin.

### SUMMARY OF THE INVENTION

The present invention details a novel process for the preparation of substantially pure 4-Fluoro-α-[2-methyl-1-oxopropyl]γ-oxo-N-β-diphenylbenzene butane amide (Formula I). 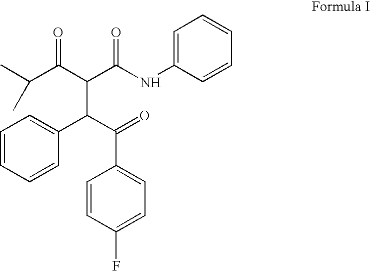
by reacting a compound of formula II 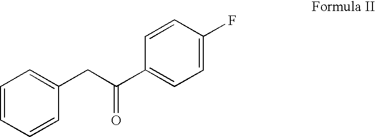
with a compound of formula III 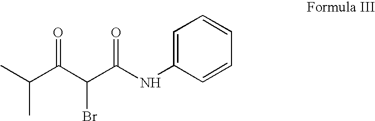
in presence of a base. Preferably the base is sodium carbonate and or a mixture of sodium carbonate and diisopropyl ethylamine.

### DETAILED DESCRIPTION OF THE INVENTION

4-Fluoro-α-[2-methyl-1-oxopropyl]γ-oxo-N-β-diphenylbenzene butane amide (Formula I) is an important intermediate for the preparation of many drug molecules especially, HMG Co-A reductase inhibitors. The HMG Co-A reductase inhibitors are useful as inhibitors of the enzyme 3-hydroxy-3-methylglutaryl-coenzyme A reductase (HMG CoA reductase) and are thus useful as hypolipidemic or hypocholesterolemic agents.

The process of the present invention in its first aspect is a new, improved, economical, commercially feasible and clean method for preparing intermediate used for the preparation of HMG CoA reductase inhibitors.

The instant invention discloses a process for the preparation of substantially pure compound of formula I 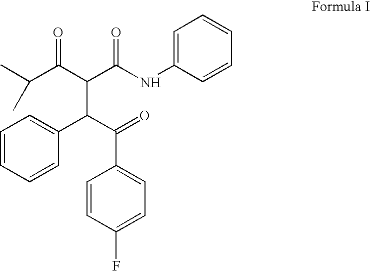
comprising of reacting a compound of formula II 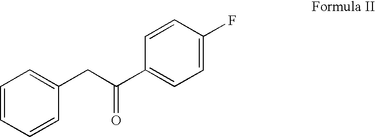
with a compound of formula III 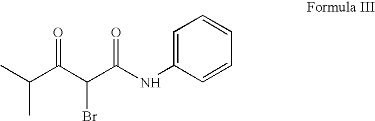
in presence of a base.

The process where the base selected from sodium carbonate, potassium carbonate, cesium carbonate diisopropyl ethyl amine, triethyl amine or a suitable mixture of two or more these.

The process where the compound of Formula I is further processed to get substantially pure atorvastatin.
Substantially pure compound of Formula I,

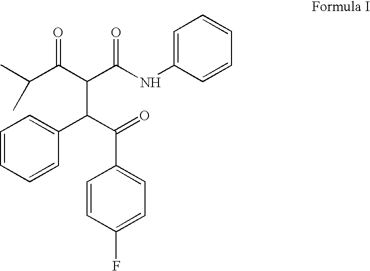

Substantially pure compound of Formula I, containing less than 0.2% of α-[2-methyl-1-oxopropyl]γ-oxo-N-β-diphenylbenzene butane amide.

Substantially pure compound of Formula I, containing less than 0.1% of difluoro α-[2-methyl-1-oxopropyl]γ-oxo-N-β-diphenylbenzene butane amide.

The present invention has following advantages over known method:
1. Clean process
2. Economic.
3. Industrially scalable.
4. The reagents used are non-hazarous, easily available and economic.
5. Yields substantially pure product, almost free of impurities like α-[2-methyl-1-oxopropyl]γ-oxo-N-β-diphenylbenzene butane amide.
6. The substantially pure compound of Formula I can be further processed to get substantially pure atorvastatin, almost free of impurities like desfloro atorvastatin.

The following non-limiting examples illustrate the inventors' preferred method for preparing the compounds of the invention.

### EXAMPLES

### Example 1

### Preparation of 4-Fluoro-α-[2-methyl-1-oxopropyl]γ-oxo-N-β-diphenylbenzene butane amide:

To a solution of 1-(4-Fluoro-phenyl)-2-phenyl-ethanone (1.5 g) in DMF (20 ml) sodium carbonate (2.5 g) was added and stirred for 15 minutes. 2-Bromo-4-methyl-3-oxo-pentanoic acid phenylamide (2 g) was added to the reaction mixture and stirred for 18 h. The reaction mixture was further stirred at about 90° C. for 5 hours. After cooling the reaction mixture to room temperature water (100 ml) was added and extracted the mixture with ethyl acetate (2×20 ml). The combined organic layer was washed with water and brine and concentrated. The residue was dissolved in hot isopropyl alcohol (15 ml) and cooled to room temperature. The product was filtered and dried. Yield: 1.5 g. The product was analyzed by HPLC and found that content of α-[2-methyl-1-oxopropyl]γ-oxo-N-β-diphenylbenzene butane amide (desfluro derivative of compound of formula I) was 0.01%

### Example 2

### Preparation of 4-Fluoro-α-[2-methyl-1-oxopropyl]γ-oxo-N-β-diphenylbenzene butane amide:

To a suspension of sodium carbonate (5 g) and diisopropyl ethylamine (16 ml) in DMF (100 ml), 1-(4-Fluoro-phenyl)-2-phenyl-ethanone (10 g) was added and stirred for 30 minutes. Further bromo-4-methyl-3-oxo-pentanoic acid phenylamide (13.5 g) was added and stirred at room temperature for 18 hours.

Subsequently diisopropyl ethylamine (8 ml) and sodium carbonate (2.5 g) were added and stirred for 10 minutes. Further bromo-4-methyl-3-oxo-pentanoic acid phenylamide (3.3 g) was added and stirred at room temperature for 5 hours. The reaction mixture was refluxed at 90-95° C. for 6 hours. After cooling the reaction mixture to room temperature, water (200 ml) was added and the contents were extracted with ethyl acetate (250 ml). The organic layer was washed with water and concentrated. The residue was re-crystallized from isopropyl alcohol to yield 4-Fluoro-α-[2-methyl-1-oxopropyl]γ-oxo-N-β-diphenylbenzene butane amide. Yield: 12 g

The product was analyzed by HPLC and found that content of α-[2-methyl-1-oxopropyl]γ-oxo-N-β-diphenylbenzene butane amide (desfluro derivative of compound of formula I) was 0.05%.

We claim:

1. A process for the preparation of substantially pure compound of formula I 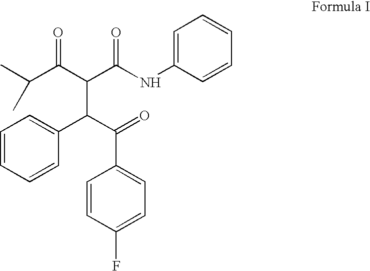
comprising of reacting a compound of formula II 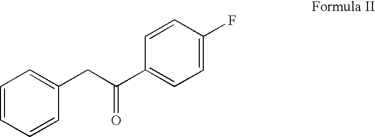
with a compound of formula III 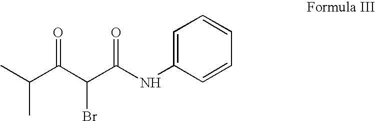
in presence of a base.

2. A process of claim 1, wherein the base selected from sodium carbonate, potassium carbonate, cesium carbonate diisopropyl ethyl amine, triethyl amine or a suitable mixture of two or more these.

3. A process of claim 1, wherein the compound of Formula I is further processed to get substantially pure atorvastatin.

4. Substantially pure compound of Formula I,

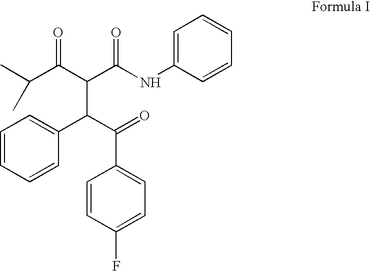

5. Substantially pure compound of claim 4, containing less than 0.2% of α-[2-methyl-1-oxopropyl]γ-oxo-N-β-diphenylbenzene butane amide.

6. Substantially pure compound of claim 4, containing less than 0.1% of difluoro α-[2-methyl-1-oxopropyl]γ-oxo-N-β-diphenylbenzene butane amide.
